(19) United States
(12) Patent Application Publication
(10) Pub. No.: US 2004/0194786 A1
(43) Pub. Date: Oct. 7, 2004

(54) Condom insert

(76) Inventor: Eric K. Manning, Dallas, TX (US)

Correspondence Address: Eric K. Manning, 206 W. Tenth St., Dallas, TX, 75208, US

(21) Appl. No.: 10/047,303

(22) Filed: Jan. 14, 2002

Related U.S. Application Data
(63) Continuation-in-part of application No. 09/262,654, filed on Mar. 4, 1999, now Pat. No. 6,298,852.

(51) Int. Cl. A61F 6/04; A61F 6/02

(52) U.S. Cl. 128/844; 128/842

(57) ABSTRACT

An improved condom accessory that maybe used with or without a condom that absorbs and eliminate sperm, comprising a mixture of prepolymer, spermicide, water and/or medication, with said condom accessory having means of strengthening mesh material embedded into the body. Various methods of attachment are used to securely hold the condom accessory in place, to prevent dislodging. In case dislodging occurs the condom accessory has a safety string as a method of retrieval from the women's vagina.

### CROSS-REFERENCE TO RELATED APPLICATION

This present application is a continuation-in-part of application Ser. No. 09/262,654, filed Mar. 4, 1999

### BACKGROUND OF THE INVENTION

Condoms have been used as a form of birth control and protection from sexually transmitted diseases for many years. The condom is a thin protective sheath for the penis used to prevent venereal infection or as a contraceptive. The condom forms a sheath around a shaft of a penis. The condom is typically made from a non-porous material preventing the transfer of semen to a woman, and in most cases, prevents pregnancy. Materials include synthetic rubbers, latex and sheepskin.

However, there are disadvantages to using condoms during sexual intercourse. Since the condom fits over the entire length of the penis, a significant reduction in sensation is noticed by the man during sexual intercourse. Another disadvantage is that, on numerous occasions, the condom can slip towards the top of the penis during the act of intercourse, causing the leakage of semen from the base of the condom. The constriction band used to hold a condom in place on the penis is typically located at the base of the condom. Since the constriction band has only the base to grasp, the condom can slip or slide during movement associated with sexual intercourse. This could allow the transfer of semen from a man to the women.

Thus, it would be a distinct advantage to have an improved condom/contraceptive device which provides increased sensation during sexual intercourse. Additionally, it would be advantageous to have an improved condom/contraceptive device which does not leak, nor prevent the transfer of semen during intercourse. It is an object of the present invention to provide such an improved condom/contraceptive device.

### BRIEF DESCRIPTION OF THE DRAWINGS

The invention will be better understood and its numerous objects and advantages will become more apparent to those skilled in the art by reference to the following drawings, in conjunction with the accompanying specification, in which:

FIG. 1 is a perspective view illustrating a traditional condom;

FIG. 2 is a perspective view illustrating a penis extension;

FIG. 3 is a side view of a penis extension inside a traditional condom;

FIG. 4 is a perspective view illustrating a round penis extension;

FIG. 5 is a perspective view illustrating a flat penis extension;

FIG. 6 is a perspective view illustrating a open penis extension;

FIG. 7 is a side view of a open penis extension inside a traditional condom;

FIG. 8 is a side view of a flat penis extension inside a traditional condom;

FIG. 9 is a perspective view illustrating adhesive tape in a unrolled state;

FIG. 10 is a perspective view illustrating rolled adhesive tape;

FIG. 11 is a perspective view illustrating a safety band;

FIG. 12 is a side view of a penis extension with safety band (penis not shown);

FIG. 13 is a side view of a penis extension with adhesive tape (penis not shown);

FIG. 14 is a side view of a penis extension with safety string;

FIG. 15 is a side view of a penis extension with over-flow canal;

FIG. 16 is a perspective view illustrating mesh; and

FIG. 17 is a perspective view illustrating a safety strap.

### DETAILED DESCRIPTION OF THE INVENTION

The present invention is an improved condom/contraceptive device providing improved sensation for the female during sexual intercourse, also preventing leakage and transfer of semen.

FIG. 1 is a perspective view illustrating a traditional condom 1. The traditional condom 1 includes a main body 3, an opening 5, and a elastomeric band 7. The entire traditional condom 1 is made of a non-porous material (e.g., rubber, latex, polyurethane, or lambskin) preventing the transmission of semen during intercourse. The main body 3 is tube-shaped for covering a entire penis.

At the base of the main body 3 is the circular opening 5. Surrounding the opening 5 is the elastomeric band 7. The elastomeric band 7 is an annular restricting band made of an elastomeric material for firmly grasping the penis for the purpose of holding the condom in place on the penis.

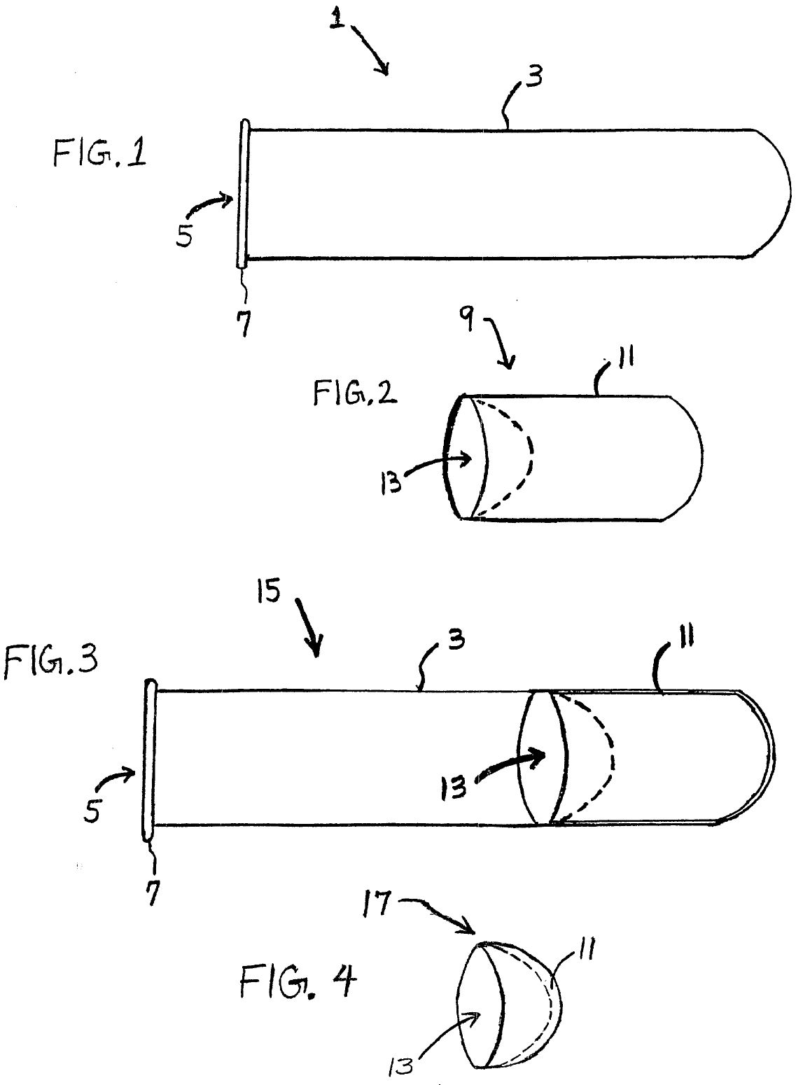

FIG. 2 is a perspective view illustrating the penis extension 9, includes a main body 11, and a opening 13.

The main body 11, maybe manufactured with a percentage of prepolymer, spermicide, water and/or various medication. Thus spermicide and/or various medication may be water or chemical activated, before use. The spermicide and/or various medication may be time released. The spermicide and/or various medication may be released from the main body 11, during sexual intercourse, into the female vagina and cervix to prevent transmission of disease and the occurrence of pregnancy. The main body 11, also absorbs, traps and eliminate sperm.

The main body 11, maybe formed from porous, non-porous materials, and/or manufactured from solid, absorbent, liquid, or dry materials either singularly or in combination with such materials as sponges (soft and hard), cloth, synthetic cloth, cotton, plastic, plastic composites, prepolymer, spermicide, rubber, latex, polyurethane, epidermal tissue, and skin. The penis extension 9, may also be bactericidal, hypoallergenic, mycocidal, spermicidal, and viralcidal.

At the base of the main body 11, is the circular opening 13. The opening 13 is half-moon shaped to surround the tip of the penis, to prevent any bends between the penis, and the main body 11. The main body 11, may consist of a tubular shaft all or in part to encompass a male penis. The tubular shaft of the main body 11, may culminate with the circular opening 13. The main body 11, may cover partial or the entire penis. The main body 11, may include adhesive or glue along the inner walls, to securely attach the penis extension 9, to the penis. By clearly attaching the main body 11, to a male penis, this would prevent back flow or slippage. The main body 11, may also be inner-lined all or in part with a thin smooth-sheath (not shown in drawings) of perforated or non-perforated latex, polyurethane, plastic, plastic composites, prepolymer, rubber, or material used to manufacture a synthetic sponge. This smooth-sheath of lining would cause some slippage giving the male a heightened sense of sensation, also allowing semen to flow pass the perforated material, thus being absorbed by the main body 11. The main body 11, may be used alone or in part with a traditional condom 1.

The main body 11 may mimic the male penis or tube-shaped for enhancing or extending the head and shaft of the penis. By enhancing the size of the male penis, this would give some women optimal pleasure, with size ranging from 0.1-8 inches either in length, width, or diameter. The main body 11, may also incorporate a round, bulbous, oblong, oval or various shapes at the aft end of the penis extension 9. The length of the main body 11 may very depending on preference, and or materials used to manufacture.

The main body 11, may also include a incision ¾ the length of the main body 11, for application purpose. This would allow the user less application time. The main body 11, may also be rolled onto itself for packaging purpose, or in a ready to use state.

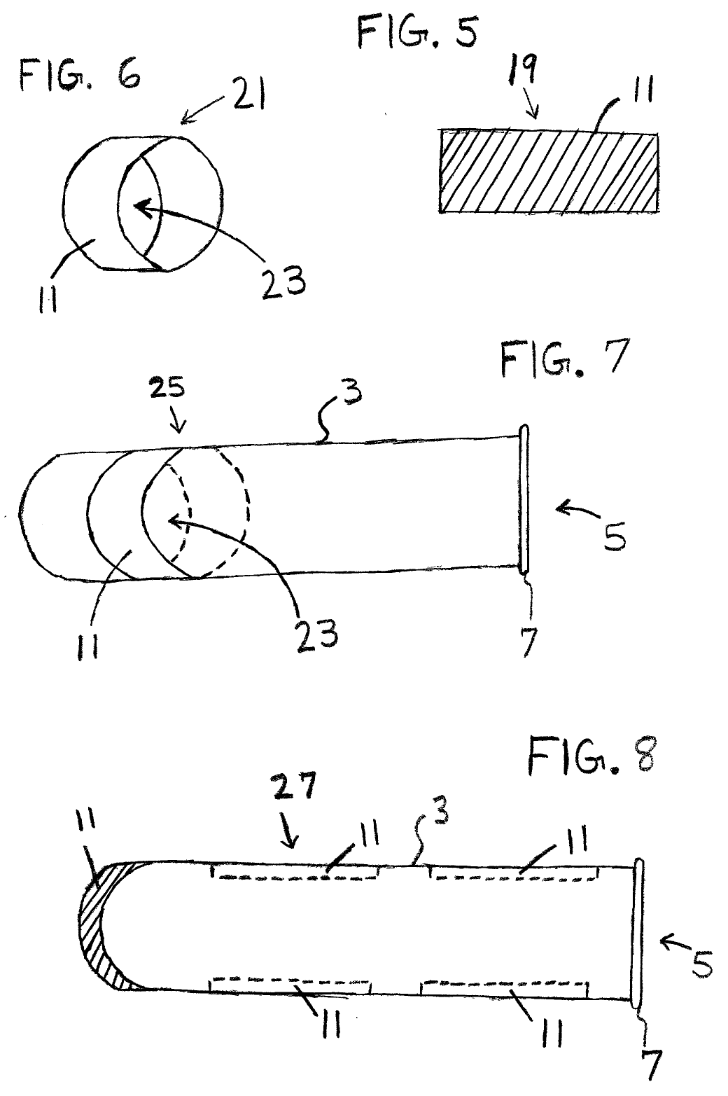

FIG. 3 is a side view of a penis extension 9, inside a traditional condom 1, thereby creating the extension condom 15. The extension condom 15, includes the main body 11 (as seen in FIG. 2, 4), a main body 3 (as seen in FIG. 1) in which a mandrel is configured to manufacture the main body 3, in which it will be able to encompass a penis and a penis extension 9. The main body 3, are from a group of singularly or in combination, consisting of rubber, latex, polyurethane, plastic, plastic composites, and sheepskin. The extension condom 15, includes a opening 13 (as seen in FIGS. 2 & 4), a opening 5 (as seen in FIG. 1), and a elastomeric band 7 (as seen in FIG. 1).

The elastomeric band 7 firmly grasps the penis for the purpose of holding the extension condom 15, firmly in place.

During sexual intercourse the penis extension 9 extends the head and shaft of the penis, also functioning as a means of absorption, and thus preventing leakage, and transfer of fluid during sexual intercourse. The penis extension 9, is primarily used as a secondary safety precaution if the traditional condom 1, tear, slip or break.

The penis extension 9, may also be bonded or attached to the traditional condom 1, by glue or adhesive, whereas the adhesive is bactericidal, hypoallergenic, mycocidal, spermicidal, and viralcidal. The adhesive is also manufactured from totally organic substances.

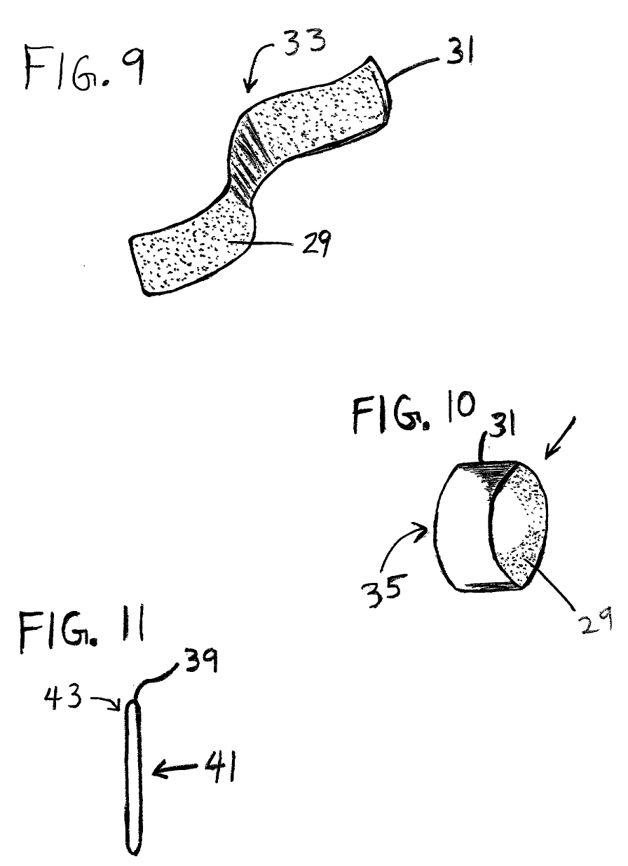

FIG. 4 is a perspective view illustration of a round penis extension 17. FIG. 4 is a example of FIG. 2, in it's round shape. FIG. 4 has all or part of the make-up and functions of FIG. 2 (e.g., FIG. 2).

FIG. 5 is a perspective view illustrating a flat penis extension 19. The flat penis extension 19, includes a main body 11. The main body 11, is flat with a flexible, absorbent body, having a angular edge. The angular edge of the main body 11, allows a method of fast and easy insertion for a male penis, during application. The main body 11, consist of all or part of the make-up and functions of the penis extension 9, as described in FIG. 2 (e.g., FIG. 2).

FIG. 6 is a perspective view illustrating a open penis extension 21. The open penis extension 21, include a main body 11, a opening 23. The opening 23, goes through and through the main body 11. The main body 11, has all or part of the make-up and functions as described in FIG. 2 (e.g., FIG. 2).

FIG. 7 is a side view of a open penis extension 21, inside a traditional condom 1, thereby creating the open extension condom 25. The open extension condom 25, includes a main body 3 (as seen in FIG. 1), a main body 11 (as seen in FIG. 2), and a opening 23, thereby creating the open extension condom 25.

The main body 11, maybe positioned through-out the traditional condom 1.

FIG. 8 is a side view of a flat penis extension condom. The flat penis extension condom 27, includes a main body 11, a main body 3, and a opening 5, thereby creating the flat penis extension condom 27.

FIG. 9 is a perspective view illustrating unrolled adhesive tape. The adhesive tape consist of adhesive 29, and a main body 31 thereby creating unrolled adhesive tape 33.

The adhesive 29 is self attaching and may include adhesive on both or either side of the main body 31. The adhesive 29 may also be in the form of glue or other means. The adhesive 29, is bactericidal, mycocidal, spermicidal, viralcidal and hypoallergenic. The adhesive 29 may also be manufactured from totally organic substances.

The main body 31, may very in size, having the characteristics of medical tape, and it may also be formed from prepolymer, thus becoming absorbent.

FIG. 10 is a perspective view illustrating rolled adhesive tape. The rolled adhesive tape 37, includes adhesive 29, a main body 31, and a opening 35, whereas the opening is created by overlapping the main body 31. The main body 31, maybe manufactured from a medical grade material.

FIG. 11 is a perspective view illustrating a safety band. The safety band 43, includes a main body 39, and a opening 41.

The main body 39, is a annular restricting band made of an elastomeric material The opening 41, is through and through which enables the safety band 43, to grasp a male penis and a penis extension 11.

FIG. 12 is a side view of a penis extension 11, with a safety band 39, thereby creating the main body 45. The main body 45, includes a penis extension 11, a safety band 39, a opening 13, and a opening 41.

The safety band 39, fits on top the penis extension 11, to securely hold the penis extension 11, on to a penis (not shown). The penis extension 11, may also be molded with a placement indention throughout, for placement of the safety band 39. The placement indention maybe mirrored or embedded around the penis extension 11. Also see FIGS. 2, 3 & 11 (e.g., FIGS. 2, 3, & 11).

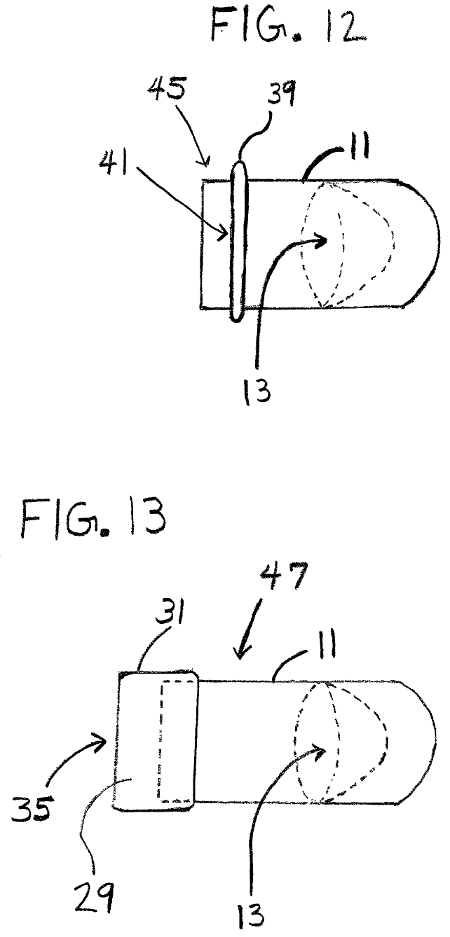

FIG. 13 is a side view of a penis extension 11, with adhesive tape 31, thereby creating the main body 47. The main body 47, includes a penis extension 11, adhesive tape 31, a opening 13, a opening 35, and adhesive 29.

The adhesive tape 31, fits halfway on top of the penis extension 11, and halfway on a male penis. This method securely holds the main body 47, in place during sexual intercourse.

FIG. 14 is a side view of a penis extension 11, with safety string 51, thereby creating the main body 49. The main body 49, includes a penis extension 11, a opening 13, and a safety string 51.

The safety string 51, is bonded to the prepolymer during or after the manufacturing process. Moments before closing the mold, a cloth like string is placed into the mixture of prepolymer, water, sulfur, spermicide and/or other medication. If the safety string 51, is bonded after curing occurs, it may be attached by glue or adhesive. The safety string 51, has a pull tab located at the opposite end. The safety string 51, is used as a safety mechanism or method of retrieval from the female vagina, if the condom tears, breaks, or slip causing the penis extension 11, to dislodge into the female vagina. This would allow the female or the user to retrieve the penis extension 11, with very little complication. The safety string 51, fits along the shaft of the penis and along the inner lining of the traditional condom 1. The pull tab of the safety string 51, hangs just outside the elastomeric band 7, of the traditional condom 1. If the penis extension 11, is used without a traditional condom 1, the safety string 51, maybe held in place by adhesive 31, or a safety band 39, along the shaft of a male penis.

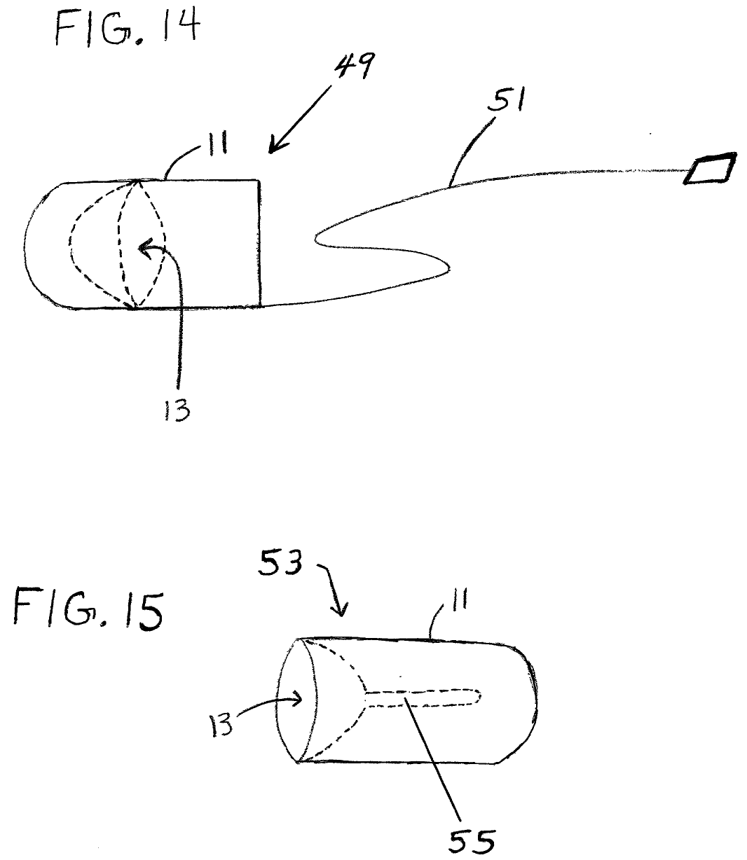

FIG. 15 is a side view of a penis extension 11, with a over-flow canal 55, thereby creating the main body 53. The main body 53, includes a penis extension 11, a opening 13, and a over-flow canal 55.

The over-flow canal 55, maybe used to absorb an abundant amount of ejaculation quicker, to prevent seepage. One or more over-flow canal 55, may culminate with the opening 13, and placed throughout the penis extension 11, in various directions.

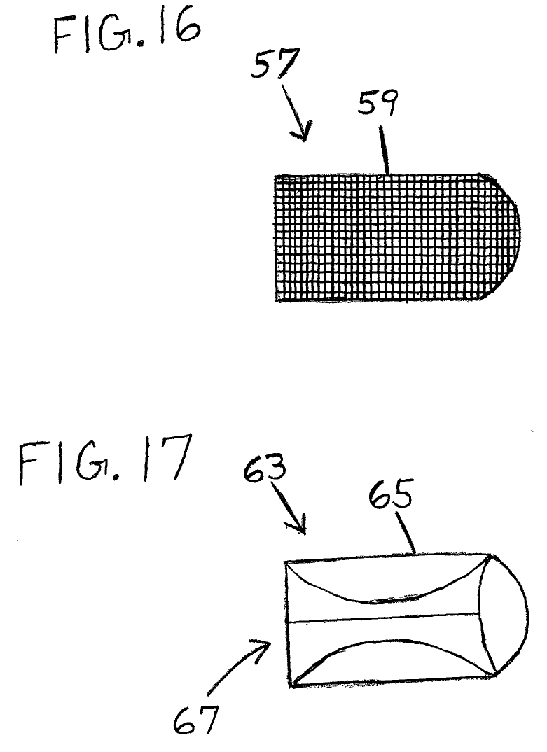

FIG. 16 is a perspective view illustrating mesh 57. The mesh 57, includes a main body 59.

The main body 59, may consist of a tightly woven material that acts as a strengthening tool, to make the penis extension 11 more tear resistant. The main body 59, is flat and maybe shape to any configuration to be place in the mold with the mixture of prepolymer. After the curing process the main body 59, forms and then becomes embedded within the penis extension 11.

The safety sting 51, of FIG. 14 may also be attached to the mesh 57, before the molding process.

FIG. 17 is a perspective view illustrating a safety strap 63. The safety strap 63, includes a main body 65, and a opening 67.

The main body 65, may include one or more elastomeric band in either direction, made to grasp and hold a penis extension 11, on a male penis. The main body 65, is a annular restricting band, made of an elastomeric material (e.g., rubber, latex, prepolymer, and polyurethane).

The opening 67, is used to encompass the penis extension 11, a male penis, thus securely holding the penis extension 11, in place.

FIG. 1-17 may consist of all or part of each other, or may have all or part of the same make-up, and furthermore maybe used in conjunction with one another.

What is claimed:

1. An improved condom accessory that covers the entire or a portion of the penis, that maybe used with or without a condom, comprising a mixture of prepolymer, spermicide, water, and/or medication, with said condom accessory having strengthening mesh material embedded in the body, with various methods of attachments to prevent dislodging, and with said condom accessory having a safety string as a method of retrieval, in case of dislodging into the female vagina, also having a safety strap that hold the condom accessory in place.

2. The improved condom accessory of claim 1, wherein the penis extension is manufactured from solid, absorbent, liquid, or dry material either singularly or in combination with such materials as sponge (soft or hard), cloth, cotton, synthetic cloth, plastic composites, prepolymer, spermicide, rubber, latex, polyurethane, epidermal tissue, endoderm tissue, smooth muscle tissue, skin, and/or various medication, that may mimic a male penis, or various shapes (e.g., round, bulbous, oblong, oval, and tube shaped).

3. The improved condom accessory of claim 1, wherein the penis extension is porous and approximately 0.1-8 inches in length, width, or diameter.

4. The improved condom accessory of claim 1, wherein the penis extension is non-porous and approximately 0.1-8 inches in length, width, or diameter.

5. The improved condom accessory of claim 1, whereas the spermicide is water activated.

6. The improved condom accessory of claim 1, whereas the spermicide is chemically activated.

7. The improved condom accessory of claim 1, whereas the spermicide maybe time released.

8. The improved condom accessory of claim 1, whereas the medication is water activated.

9. The improved condom accessory of claim 1, whereas the medication is chemically activated.

10. The improved condom accessory of claim 1, whereas the medication maybe time released.

11. The improved condom accessory of claim 1, wherein the penis extension absorbs sperm.

12. The improved condom accessory of claim 1, wherein the penis extension traps sperm.

13. The improved condom accessory of claim 1, wherein the penis extension eliminate sperm.

14. The improved condom accessory of claim 1, wherein the penis extension is bonded to the condom by glue.

15. The improved condom accessory of claim 1, wherein the penis extension is bonded to the condom by adhesive.

16. The improved condom accessory of claim 1, wherein the penis extension maybe bonded by means of glue to the male penis.

17. The improved condom accessory of claim 1, wherein the penis extension maybe bonded by means of adhesive to the male penis.

18. The improved condom accessory of claim 1, wherein the penis extension maybe inner-lined with a perforated or non-perforated thin smooth-sheath all or in part.

19. The improved condom accessory of claim 18, whereas the thin smooth-sheath maybe manufactured all or in part with latex, polyurethane, plastic, plastic composites, prepolymer, rubber, or material used to manufacture a synthetic sponge.

20. The improved condom accessory of claim 1, wherein the penis extension may include a incision ¾ the length, along the body for application purpose.

21. The improved condom accessory of claim 1, wherein the penis extension maybe rolled onto itself for packaging purpose.

22. The improved condom accessory of claim 1, whereas the penis extension maybe flat in shape.

23. The improved condom accessory of claim 22, wherein the flat penis extension may have a flexible body with a angular edge, allowing easy, faster application time.

24. The improved condom accessory of claim 1, wherein the penis extension has a opening through and through.

25. The improved condom accessory of claim 1, whereas the penis extension maybe positioned throughout the inner lined wall of the traditional condom.

26. The improved condom accessory of claim 1, whereas the penis extension maybe positioned throughout the outer lined walls of the traditional condom.

27. The improved condom accessory of claim 1, wherein the penis extension may incorporate self attaching adhesive tape that may have adhesive on one or more side.

28. The improved condom accessory of claim 27, wherein the adhesive may consist of glue or like means.

29. The improved condom accessory of claim 28, wherein the adhesive is bactericidal.

30. The improved condom accessory of claim 28, wherein the adhesive is mycocidal.

31. The improved condom accessory of claim 28, wherein the adhesive is spermicidal.

32. The improved condom accessory of claim 28, wherein the adhesive is viralcidal.

33. The improved condom accessory of claim 28, wherein the adhesive is hypoallergenic.

34. The improved condom accessory of claim 28, manufactured from totally organic substances.

35. The improved condom accessory of claim 28, wherein the adhesive is manufactured from a medical grade material.

36. The improved condom accessory of claim 1, wherein the penis extension may incorporate a safety band.

37. The improved condom accessory of claim 1, wherein the penis extension may include a placement indention.

38. The improved condom accessory of claim 37, wherein the safety band is placed in the placement indention.

39. The improved condom accessory of claim 37, wherein the placement indention maybe mirrored throughout the penis extension.

40. The improved condom accessory of claim 37, wherein the placement indention is embedded throughout the penis extension.

41. The improved condom accessory of claim 1, wherein the penis extension may incorporate a safety string with a pull tab.

42. The improved condom accessory of claim 41, wherein the safety string is bonded to the mixture of prepolymer during the manufacturing process.

43. The improved condom accessory of claim 41, wherein the safety string is bonded to the penis extension by glue.

44. The improved condom accessory of claim 41, wherein the safety string is bonded to the penis extension by adhesive.

45. The improved condom accessory of claim 1, wherein the penis extension may incorporate a over-flow canal.

46. The improved condom accessory of claim 1, wherein the penis extension may incorporate mesh.

47. The improved condom accessory of claim 1, wherein the penis extension may incorporate a safety strap.
